(19) United States
(12) Patent Application Publication
(10) Pub. No.: US 2024/0269339 A1
(43) Pub. Date: Aug. 15, 2024

(54) DISSOLUTION RESISTANT TISSUE ADHERENT DRESSING APPLICATION AND ITS DELIVERY

(71) Applicant: TRICOL BIOMEDICAL, INC., Portland, OR (US)

(72) Inventors: Kenneth E. PERRY, Bainbridge Island, WA (US); Kenneth P. PERRY, Bainbridge Island, WA (US); MacKayla CAROLAN, Portland, OR (US); Madison BUTLER, Portland, OR (US); Shalini GAUTAM, Hillsboro, OR (US); Simon John MCCARTHY, Portland, OR (US)

(21) Appl. No.: 18/568,202

(22) PCT Filed: Jun. 8, 2022
(86) PCT No.: PCT/US2022/032665

§ 371 Date: Dec. 7, 2023

Related U.S. Application Data
(60) Provisional application No. 63/208,209, filed on Jun. 8, 2021.

(51) Int. Cl. A61L 15/28; A61F 15/00; A61L 15/42; A61L 15/44; A61L 15/58

(52) U.S. Cl. CPC A61L 15/28; A61F 15/005; A61L 15/425; A61L 15/44; A61L 15/58; A61L 2400/04

(57) ABSTRACT

The present invention relates to a dissolution resistant, biocompatible, foldable, thin profile, low mass and high surface area, chitosan dressing, optionally modified with iron, and suitable for treating bleeding in a physiological environment, e.g. gastrointestinal tract. The characteristics and structures of the chitosan dressing are provided. Devices and systems for delivery of dressing to a target tissue site are provided. Methods of making and using the chitosan dressing are also provided.

### GOVERNMENT LICENSE RIGHTS

This invention was made with Government support under R44DK104564 awarded by National Institute of Diabetes and Digestive and Kidney Disease. The Government has certain rights in the invention.

### BACKGROUND

### Technical Field

This disclosure relates to the field of chitosan materials comprising catechol modified chitosan and uses thereof.

### Description of the Related Art

Prolonged bleeding, with its associated risks in mortality and morbidity, remains a serious problem in the gastrointestinal (GI) tract. Techniques and devices that could provide for rapid bleeding control in gastrointestinal bleeding (GIB) for both upper gastrointestinal bleeding (UGIB) and lower gastrointestinal bleeding (LGIB) are needed. Although there have been advances in bleeding control using advanced dressings for applications outside of GIB bleeding none of these advances have yet translated to the unique conditions of the gastrointestinal tract and especially the upper gastrointestinal tract where delivery, adhesion, enzyme activity and acidity considerations are highly challenging.

Gastrointestinal bleeding (GIB) is a common presentation to the emergency department. According to the U.S. Department of Health and Human Service, from 2000 to 2014, there was an average of over 350,000 discharges from gastrointestinal hemorrhage annually. In the U.S., the direct hospital cost in 2010 due to GIB exceeded $1.1 billion. Upper GIB (UGIB), defined as gastrointestinal bleeding proximal to the ligament of Treitz, is approximately five times more common than lower GIB (LGIB). Acute UGIB is a potentially life-threatening emergency that necessitates prompt assessment, resuscitation and appropriate medical and endoscopic management. Despite recent advances in management of GIB in western countries, the mortality rate of acute UGIB has not significantly improved, and remains as high as 10-14%. The major cause of death after GIB is death secondary to cardiorespiratory complications, which is not surprising given the burden of comorbidities in such patients; death due to uncontrollable hemorrhage is reported to account for between 20% and 25% of cases [5, 6]. While little can be done to correct comorbidities urgently, more effective and rapid bleeding control will allow significant reductions in the incidence of UGIB related morbidity and mortality. In general, the most common causes of acute UGIB are peptic ulcers, gastro-esophageal varices, Mallory-Weiss tears and erosive esophagogastritis. Nonvariceal upper gastrointestinal bleeding (NVUGIB) encompasses all causes of UGIB except bleeding esophageal or gastric varices. The incidence of peptic ulcer disease has decreased because of the development and utilization of proton pump inhibitors as well as the identification, treatment and eradication of Helicobacter pylori in individual patients. Despite decreased peptide ulcer incidence, mortality among NVUGIB patients ranges from 3-4%. While rarely life threatening, gastric malignancies can lead to friable tissue with diffuse bleeding that is difficult to address with traditional physical hemostatic methods (clips, bands, ligation) or cautery.

Current endoscopic management of patients with acute UGIB includes thermal therapy (e.g., bipolar electrocoagulation, heater probe, monopolar electrocoagulation, argon plasma coagulation, and laser), injection (epinephrine, sclerosants (e.g., absolute ethanol, polidocanol, and ethanolamine)), thrombin or fibrin glue (thrombin plus fibrinogen)), and clips.

In general, the majority of patients with bleeding peptic ulcers, hemostasis is achieved with combination of the above endoscopic therapeutic modalities. However, there remains a subset of patients, approximately 5%, in which endoscopic treatments are not sufficient for hemostasis and thus require interventional radiology or surgical interventions.

Endoscopic therapy fails for a variety of reasons including poor visibility of lesion due to active pulsating bleeding, difficult anatomic location of lesion for endoscopy, maximal therapy with currently available tools, and severe coagulopathy.

### BRIEF SUMMARY

Although existing tools in the United States readily control a significant portion of UGIB, there remains unmet need for the low risk dressings and devices for the delivery of the dressings of the subject invention that provides rapid control of brisk arterial bleeding. Broad application of the subject invention will enable significant reduction in morbidity and mortality in gastrointestinal bleeding treatment with concomitant reduction in associated health care expenditure.

The subject chitosan gastrointestinal hemostatic dressing (CGHD) of the invention is amenable to use in all gastrointestinal bleeding applications and may be delivered by, for example, wire delivery through a standard endoscopic working channel (≤3.8 mm diameter) or by balloon catheter delivery. The subject CGHD invention will provide an opportunity to address or mitigate deficiencies with current modalities, such as clipping, thermal coagulation and injection, which necessitate pinpoint accuracy and which is challenging under impaired visibility of brisk bleeding conditions.

The present invention comprises compositions, methods of using the compositions, methods of making the compositions, and systems and devices for delivering the compositions.

In certain embodiments, the chitosan gastrointestinal hemostatic dressing (CGHD) comprises a catechol modified chitosan, wherein the dressing is hemostatic and has a thickness that is 500 microns or less.

In certain embodiments, the catechol modified chitosan (Cs-Cat) is formed by N-acylation of the C-2 amine on the chitosan glucosamine by 3,4-dihydroxyhydrocinnamic acid (alternatively named 3-(3,4-Dihydroxyphenyl) propionic acid, Hydrocaffeic acid)). Alternatively, the chitosan N-acylation to produce a catechol modified chitosan (Cs-Cat) may include but not be limited to a modification with one of a 3,4-Dihydroxycinnamic acid (caffeic acid); a trans-3,4-Dihydroxycinnamic acid (trans-caffeic acid); and a 3,4-Dihydroxyphenylacetic acid (DOPAC, Homoprotocatechuic acid). Catechol reactants, including but not limited to 3,4-dihydroxyhydrocinnamic acid, and 3,4-Dihydroxycinnamic acid, 3,4-Dihydroxyphenylacetic acid are represented commonly as catechol reactant moiety or CatH. The catechol reactant moiety minus a hydrogen (Cat) is added to the C-2 amine on the chitosan glucosamine following an N-acylation reaction to produce catechol chitosan (Cs-Cat).

In some embodiments, the dressing is a releasable iron-enhanced catechol modified chitosan dressing (IECD). The iron-enhanced catechol modified chitosan dressing comprises an iron salt. In some embodiments, the iron salt is ferric chloride. The addition of iron to the chitosan catechol synthesis was found to be able to provide improved dressing synthesis with enhanced reproducibility in lot-to-lot preparations. In certain embodiments, the dressing is a composite thin dressing comprising a catechol modified chitosan and iron-enhanced catechol modified chitosan.

In some embodiments, the dressing has a thickness that is about 40-140 microns. The dressing described herein is foldable, resistant to swelling, resistant to gastric fluid digestion, tissue adhesion and has improved mechanical strength. In certain embodiments, the dressing is cross-linked.

In some embodiments, the dressing has a density that is in the range of about 0.5 g/cm³ to about 1.1 g/cm³. In certain embodiments, the dressing has a density that is in the range of: (i) about 0.5 g/cm³ to about 0.8 g/cm³; or (ii) about 0.8 g/cm³ to about 1.1 g/cm³.

In some embodiments, the dressing has an adhesive side and a non-adhesive side. The adhesive side may be provided on a first layer and the non-adhesive side may be provided on a second layer. In certain embodiments, the adhesive side adheres to a tissue surface when the dressing is wet. In a preferred embodiment, the dressing adheres to a gastrointestinal mucosa in 1 minute or less. In some embodiment, the dressing when wet is able to adhere intact to gastric mucosa in less than 30 seconds with application of light pressure. In an embodiment, the light pressure is about 50-100 g/cm².

In certain embodiments, the dressing adherence strength is greater than or equal to about 1 kPa. In some embodiments, the non-adhesive side does not adhere to a delivery device when the dressing is wet. In a preferred embodiment, the adhesive side comprises an iron-enhanced catechol modified chitosan. In some embodiments, the dressing comprises an adhesive side that interacts with an injury site, and a non-adhesive side that interacts with one of a delivery device or the adhesive side of the dressing, when the dressing is in a dry and folded or a dry and furled condition.

The CGHD described herein is resistant to dissolution. In a preferred embodiment, the dressing resists dissolution in water, saline solution, blood, or GI fluid at about 37° C. for at least about 6 hours. In certain embodiments, the dressing is not readily soluble in water, saline solution, blood, or GI fluid at about 37° C. for at least 12 hours following application. In some embodiments, the dressing is not readily soluble in water, saline solution, blood, or GI fluid at about 37° C. for: (1) at least 12 hours following application; or (2) at least 24 hours following application; or (3) both (1) and (2). In a preferred embodiment, the dressing does not increase or decrease in size by more than about 25% in length and width, or more than about 50% in thickness in the presence of water, saline solution, blood, or GI fluid at about 37° C.

In certain embodiments, the dressing can be punctured or sewn without cracking or tearing. In some embodiments, the dressing further comprises a porous surface. In certain embodiments, the porous surface provides one or more of: (i) an absorbent surface; and (ii) channels to redirect moisture away from a target tissue surface site. In some embodiments, the dressing is able to remove hydrophilic and hydrophobic biological fluids that can interfere with adhesion. In a preferred embodiment, the dressing is able to stay in place intact and stop moderate to oozing bleeding ranging from ranging from between about 1 g/min to about 30 g/min, preferably about >5 g/min.

In some embodiments, the dressing is capable of being terminally sterilized without affecting dressing characteristics. The dressing described herein is capable of being stored under controlled conditions over time without affecting dressing characteristics.

The subject dressing disclosed herein is able to be delivered intact by a balloon device, a wire device, or an endoscopic device. In some embodiments, the balloon device, the wire device, or the endoscopic device comprises a working channel having a diameter of 3.8 mm or less, and the dressing disclosed herein is delivered through the working channel. In a preferred embodiment, the dressing disclosed herein is delivered intact by a balloon device, a wire device, or an endoscopic device comprising a working channel having a diameter of 3.7 mm. In an embodiment, the dressing readily detaches from a delivery device after adherence to a target tissue site. In some embodiments, the dressing is able to resist dissolution for at least six hours after adhering to an injury site in presence of corrosive enzymes and acid environment of about pH 3. The subject dressing disclosed herein is able to seal and protect a target tissue site for up to about 12 hours, preferably up to about 24 hours, and more preferably up to about 96 hours. In a preferred embodiment, the dressing is able to achieve a controlled, slow dissolution from an attachment site over a period of time not exceeding seven (7) days.

The subject dressing can be folded or furled without cracking or tearing. The dressing may, in an open, unfurled, or unfolded condition, have an outward facing surface area that is one of about six times greater, about five times greater, or about four times greater than the outward facing surface area of that same dressing when it is in a closed, furled, or folded condition.

In some embodiments, the invention disclosed herein discloses gastrointestinal hemostatic dressing delivery devices. The gastrointestinal hemostatic dressing delivery devices described herein are amenable to use in all gastrointestinal bleeding applications and can be used to deliver and apply a dressing to a target tissue site via a narrow channel such as, for example, an endoscopic channel. The devices described herein may be used in minimally invasive procedures.

In some embodiments, the devices described herein comprise a gastrointestinal delivery system comprising a wire delivery device; and a releasable wound dressing. In certain embodiments, the wire delivery device comprises an axis, an expandable support, a dressing and, optionally, a sheath. In some embodiments, a single structure may serve as both the axis and the expandable support. For example, the device may comprise a wire base axis, a balloon catheter expandable support, and a dressing delivered through a standard endoscopic working channel having a diameter of less than or equal to 3.7 mm, or a laser-cut cylinder of nitinol or stainless steel with free ends. In a further aspect, the wire delivery device is a 4-arm-90° wire delivery (4Arm90 WD) device. In some embodiments, the wire delivery device is a basket wire delivery (Basket WD) device; or looped wire delivery (Looped WD) device.

The gastrointestinal delivery system provides for the compact delivery of a splayed high surface area dressing to a target tissue treatment site. In some embodiments, the hemostatic dressing is a chitosan gastrointestinal hemostatic dressing (CGHD). In some embodiments, the dressing is a catechol modified chitosan dressing. In a preferred embodiment, the dressing is an iron-enhanced catechol modified chitosan dressing (IECD). In some embodiments, the dressing is a composite dressing comprising catechol modified chitosan and iron-enhanced catechol modified chitosan dressing (IFCD).

In some embodiments, the invention disclosed herein discloses a system to control bleeding from a target tissue site comprising a hemostatic dressing and a delivery device. In some embodiments the hemostatic dressing comprises a catechol modified chitosan, wherein the dressing has a thickness that is 500 microns or less. In a preferred embodiment, the hemostatic dressing is an iron-enhanced catechol modified chitosan dressing (IECD) comprising an iron salt. In some embodiments, the IECD has a thickness that is about 40-140 microns.

In an embodiment, the device disclosed herein is a wire delivery device. In some embodiments, the wire delivery device comprises more than one wires. In certain embodiments, the more than one wires comprise a shape-memory alloy. In an embodiment, the shape-memory alloy is a nitinol.

In an embodiment, the wire delivery device is capable of: (i) attaching to the dressing during loading; and (ii) releasing the dressing during delivery. In some embodiments, the wire delivery device is a basket wire delivery (Basket WD) device; or looped wire delivery (Looped WD) device. In some embodiments, the delivery device has a spring loading tension between about 50-100 g during dressing delivery. In some embodiments, the delivery device has a spring loading tension between about 150-250 g during dressing delivery. In a preferred embodiment, the delivery device has a spring loading tension of 250 g. In some embodiments, the delivery device has a spring loading tension of about >300 g. In certain embodiments, the gastrointestinal delivery system is capable of fitting through one of: a channel of about 4.0 mm internal diameter or less; a channel of about 3.8 mm internal diameter or less; a channel of about 3.4 mm internal diameter or less; a channel of about 3.1 mm internal diameter or less; or a channel of about 2.8 mm internal diameter or less.

In an embodiment, an initial wire delivery (WD) prototype with i) 50-100 g spring loading; ii) 8 wire×0.24 mm OD nitinol delivery system; and iii) in the catheter reduced cross-sectional area (folded and compressed) near 0.44 mm² provided dressing space from a gastroscope channel as small as 2.8 mm diameter and demonstrated with ease control of Forrest 1a hemorrhage. In an embodiment, a wire delivery prototype with i) 150-250 g spring loading; ii) 32 wires×0.22 mm diameter nitinol closed-ended basket; and iii) in the catheter cross-sectional area >4.5 mm² (requiring a gastroscope channel near 4.0 mm diameter to accommodate the folded dressing) demonstrated >90% successful dressing delivery in control of Forrest 1a hemorrhage.

In some embodiments, the basket wire delivery (Basket WD) device comprises: (i) an expandable support comprising a close-ended basket; and (ii) a support tubing. In certain embodiments, the close-ended basket comprises multi-wire and interwoven wire spirals with spacing of at least 2 mm between adjacent wires.

In certain embodiments, the looped wire delivery (Looped WD) device comprises an expandable support comprising two looped wires offset at about 90° to each other or three looped wires offset at about 60° to each other. In some embodiments, the Looped WD further comprises a stainless steel cannula.

In some embodiments, the invention disclosed herein comprises methods of producing the hemostatic chitosan dressing. In one embodiment, the method comprises: performing synthesis with chitosan and catechol in an aqueous reaction solution; maintaining a pH of the reaction solution at or below pH 5.5; increasing the pH of the reaction solution, and controlling oxygen exposure to the reaction solution, to provide catechol oxidation and cross-linking; and drying the reaction solution. In some embodiments, the method further comprises adding an iron salt. In a preferred embodiment, the iron salt added is ferric chloride. In certain embodiments, the method comprises forming composite thin dressings of iron-enhanced catechol modified chitosan and catechol modified chitosan.

In an embodiment, the method of producing the hemostatic chitosan dressing comprises freeze drying a first aqueous solution comprising catechol modified chitosan; freeze drying a second aqueous solution comprising catechol modified chitosan; obtaining a low-density chitosan dressing with inter-connected porous structure from each of steps (a) and (b); and compressing the low-density chitosan dressing from each of steps (a) and (b); and preparing a two-layer catechol chitosan composite dressing from the compressed low-density chitosan dressing from each of steps (a) and (b). In some embodiments, the second aqueous solution comprises an iron salt, wherein the iron salt is ferric chloride, and wherein the molar ratio of iron to catechol reactant moiety (CatH) is either about 1:5 (Moderate Fe) or about 1:1 (High Fe).

In an embodiment, the method of producing the hemostatic chitosan dressing comprises freezing a first aqueous solution comprising catechol modified chitosan; and aspirating a fine mist of a second aqueous solution on to the pre-frozen first aqueous solution to create a second very thin frozen layer on, and attached to, the pre-frozen solution. The combined two layer frozen cake is then freeze dried to sublimate the ice. The low density dried sponge is then compressed to its final density. In a preferred embodiment, the first aqueous solution comprises an iron-enhanced catechol modified chitosan.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE SEVERAL VIEWS OF THE DRAWINGS

FIG. 1 shows two 4Arm90 WD prototype #1 super-elastic nitinol wire delivery (WD) device with Perspex rod handle for hand manipulation in early prototype evaluation. In catheter delivery testing, this rod handle was substituted for a wire axis distal end delivery connector attached to the two proximal wire ends of the 4Arm90 supporting axis strut.

FIG. 2 shows 4Arm90 WD prototype #3 WD demonstrating articulated central interlocked loops and absent 90° twist in the “T” ends.

FIG. 3 shows 4Arm90 WD prototype #3 WD device with 2 cm central axis strut.

FIG. 4 shows improvised stent basket shape WD formed by closing one end of the gastrointestinal stent with braid and placing a tight collar around the other end.

FIG. 5 shows rounded shape of improvised basket WD device with central rod allowing for preforming of the basket shape on expressing the improvised stent from the catheter proximal end.

FIG. 6 shows simple loop prototype WD device with 3 loops offset to each other by 60°.

FIG. 7 shows 4 Arm90 WD prototype #1 WD device with glued articulated tab attachment of an iron-enhanced catechol modified chitosan dressing prototype.

FIGS. 8A-8B show Delnet backed dressing with attached cross threads of dissolvable polyvinyl alcohol (PVA) from corner to corner on 15 mm×15 mm IECD (sandwiched between Delnet and iron-enhanced catechol modified chitosan dressing) (FIG. 8A); and 4Arm90 WD prototype #3 WD (2 cm axis strut) with Delnet backed dressing attached by tying PVA thread to WD cross-strut ends (FIG. 8B).

FIG. 9 shows pre-fold formed Type A dressing ready for applying tightly over compressed basket tip and loading inside delivery catheter.

FIG. 10 shows flattened dressing back side showing thin medical elastomer film reinforcement circle close to about 3 mm diameter in its center. Above the dressing is an about 4.4 mm ID catheter proximal end loaded with dressing over collapsed stent end.

FIG. 11 shows histogram showing temporal left to right progression of detachment time from stomach mucosa. Iron-enhanced catechol modified chitosan dressing formulations resulted in significant increase in both consistency and detachment time. The average time to dissolution for all patches containing iron considered for the study was longer than 12 hours. Swelling of iron containing prototypes in length and width met requirements and was less than 25%.

FIGS. 12A-12C show injury site with brisk bleeding (30 g/min) and spurting from laceration injury on vascular bundle (FIG. 12A); basket WD delivery of type B dressing to injury site (FIG. 12B); and Type B dressing adhered in place controlling previously brisk anticoagulated swine model Forrest 1a hemorrhage within 2 minutes of application(FIG. 12C).

FIG. 13 shows 4Arm90 WD delivery of a Type C dressing.

FIGS. 14A-14B show basket WD delivery of Type A dressing (FIG. 14A); and successfully delivered Type A dressing (FIG. 14B).

FIG. 15 shows percent success and failure among dressing types A, B and C with 12, 9 and 10 applications, respectively, including both basket and 4Arm90 WD device applications.

FIG. 16 shows average time to hemostasis in minutes by dressing types A, B and C with 9, 9 and 7 successful applications, respectively, including basket and 4Arm90 WD device applications.

FIG. 17 shows average adherence rankings of dressing types A, B and C indicating how well the dressings adhered to the tissue of the injury site and surrounding stomach mucosa (0.0 is the lowest with 4.0 the highest rank).

FIG. 18 shows Type A dressing demonstrating excellent tissue adherence to its original injury site, and little dressing change after 20 hours wrapped inside the surgically removal stomach.

FIG. 19 shows image of Type A dressing on tissue explanted 72 hours after implantation on moderately bleeding heparinized 6 mm biopsy punch upper gastrointestinal (stomach) mucosa injury.

### DETAILED DESCRIPTION

The disclosure generally relates to a biocompatible, foldable, thin profile, chitosan-based dressings, gastrointestinal hemostatic dressing (CGHD) and devices that can be used in all gastrointestinal bleeding applications to deliver and apply a dressing to a target tissue site. Suitable dressing materials may be configured to and/or capable of being passed through a channel and may comprise a dressing selected from a catechol modified chitosan, an iron-enhanced catechol modified chitosan dressing, or composite dressings comprising catechol modified chitosan and iron-enhanced catechol modified chitosan. The dressings described herein are capable of being used alone, i.e., without delivery assisted by passing through a channel.

Alternatively, the gastrointestinal dressing and delivery device systems provided herein can be used in combination with other medical devices, including but not limited to, an endoscope, such as an endoscope for gastroscopy.

The dressings described herein are characterized by one or more, or all, of the following features, such that it is: (1) able to be delivered intact by balloon or a wire device, or through endoscopic device; (2) is able to wet and adhere intact to gastric mucosa in under 30 seconds with application of light pressure; (3) has capillarity, porosity and absorbency that is able to remove hydrophilic and hydrophobic biological fluids that can interfere with adhesion; (4) is able to stay in place intact and stops oozing to hemorrhagic bleeding, e.g., a bleeding rate of between about 0.5 ml/min to about 30 ml/min, or greater; (5) is able to be released from the delivery device to allow withdrawal of the delivery device from the GI environment; (6) is able to resist detrimental rapid breakdown (<6 hours) in the corrosive enzymes and acidity (≥pH 3) of the GI environment; (7) is able to protect the gastrointestinal injury site for preferably up to 12 hours, more preferably up to 24 hours and most preferably up to 96 hours to assist with its subsequent acute healing and closure; and (8) is able to achieve a controlled, slow dissolution from the attachment site to allow for unassisted complete removal in less than seven days with the dissolved residue passing safely through the alimentary tract.

The disclosure further relates to gastrointestinal hemostatic dressing delivery devices. The devices described herein are capable of being fit through a narrow channel with a dressing before reaching a desired site, or target tissue site, in vivo and delivering the dressing to the target tissue site. The devices described herein include mechanisms to introduce the dressing into the GI environment from the end of a narrow channel. The devices also involve taking the dressing from a compact condition to a splayed condition. In some embodiments, introduction of the dressing into the GI environment from the end of a narrow channel involves introducing the dressing in a compact condition. In alternative embodiments, the introduction of the dressing into the GI environment from the end of a narrow channel involves introducing the dressing in a transition condition or a splayed condition as it emerges from the narrow channel. In some embodiments, the dressing is released from the device after contact with a target tissue site. In some embodiments, the dressing is released upon one or more of expansion of the expandable support into an expanded format and adhesion of the dressing to the target tissue site.

In some embodiments, the dressing seals a target tissue site in gastrointestinal tract. In the case of the GI tract, sealing is complicated by the gel like nature of the mucus of the stomach lining. Attachment to this gel mucus layer is important for efficacy. In some embodiments, the adhered hemostatic dressing promotes bleeding control partly by sealing the wound but also by promotion of local clot formation. For example, mucoadhesive chitosan dressings are highly effective at stopping bleeding by sealing of the target tissue site and by providing local hemostatic promoting capability.

In one embodiment, the dressing provides a unique opportunity for treatment of adherent clots, i.e., by reinforcing and promoting local healing, that could otherwise fail and cause serious hemorrhage if left untreated. Currently there is no treatment for large adherent clots found in the upper GI tract other than keeping subjects under close observation.

In some embodiments, the dressing may further comprise a pharmaceutical active agent (drug or biologic) that may be delivered and applied locally to the target tissue site.

In some embodiments, the dressing stops bleeding at the site in gastrointestinal tract. In one embodiment, the device delivers a CGHD to a target tissue site in the gastrointestinal tract. In some embodiments, the dressing comprises a catechol modified chitosan. In some embodiments, the dressing comprises an iron-enhanced catechol modified chitosan dressing. In one preferred embodiment, the dressing is a composite dressing comprising catechol modified chitosan and iron-enhanced catechol modified chitosan. In another preferred embodiment, the dressing is a single layer dressing comprising iron-enhanced catechol modified chitosan.

Preferably the catechol modified chitosan (Cs-Cat) is formed by N-acylation of the C-2 amine on the chitosan glucosamine by 3,4-dihydroxyhydrocinnamic acid (alternatively named 3-(3,4-Dihydroxyphenyl) propionic acid, Hydrocaffeic acid)). Alternatively, the chitosan N-acylation to produce a catechol modified chitosan (Cs-Cat) may include but not be limited to a modification with one of a 3,4-Dihydroxycinnamic acid (caffeic acid); a trans-3,4-Dihydroxycinnamic acid (trans-caffeic acid); and a 3,4-Dihydroxyphenylacetic acid (DOPAC, Homoprotocatechuic acid). Catechol reactants, including but not limited to 3,4-dihydroxyhydrocinnamic acid, and 3,4-Dihydroxycinnamic acid, 3,4-Dihydroxyphenylacetic acid are represented here commonly as catechol reactant moiety or CatH. The catechol reactant moiety (Cat) minus a hydrogen is added to the C-2 amine on the chitosan glucosamine following an N-acylation reaction to produce catechol chitosan (Cs-Cat).

The present disclosure also pertains to a gastrointestinal delivery device comprising: an expandable support; and a dressing. In some embodiments, the device further comprises a protective sheath. In some embodiments, the device is a wire delivery device. In some embodiments, the device is capable of fitting through a channel of about 4.4 mm internal diameter (ID) or less. In some embodiments, the device is capable of fitting through a channel of about 3.7 mm internal diameter (ID) or less. In some embodiments, the expandable support comprises more than one wire. In some embodiments, the expandable support is capable of: (i) attaching to the dressing during loading, and (ii) releasing the dressing during delivery. In some embodiments, the wire delivery device comprises a shape-memory alloy. In some embodiments, the shape-memory alloy is a nitinol. In a further aspect, the wire delivery device is selected from: 4-arm-90° wire delivery (4Arm90 WD) device; basket wire delivery (Basket WD) device; and looped wire delivery (Looped WD) device.

In a preferred embodiment, once the splayed dressing is applied to a target tissue site, it is released, and the other delivery components and mechanisms used to deliver the dressing are removed from the target tissue site. The dressing may be releasably attached to one or both of the axis and the expandable support. The expandable support may be collapsed before it is removed from the target tissue site. All device components other than the dressing may then be removed from the target tissue site.

The disclosure further relates to the use of the dressings disclosed herein for the treatment of a disease, condition, disorder, trauma, or injury. In some embodiments, the use comprises directly adhering the dressing at an injury site upon wetting, and applying pressure to the dressing for about 30 seconds. In certain embodiments, the dressing removes hydrophilic and hydrophobic biological fluids upon adherence. In some embodiments, the use further comprises leaving the dressing in place at a target tissue site. In a preferred embodiment, the dressing remains at the target site for at least 24 hours. In some embodiments, the dressing is capable of slow dissolution at the target site and dissolves completely without human intervention in seven days or less.

### EXAMPLES

The following examples are offered by way of illustration and not by way of limitation.

### Example 1

### Preparation of Iron Catechol Chitosan Dressings

The following materials were used in the preparation of iron-enhanced catechol chitosan dressings: Chitosan B: Primex ChitoClear 43000, TM 4167, MW=110-150 kDa, Brookfield viscosity in 1.0% w/w chitosan solution in 1.0% acetic acid at 25° C. and spindle LV1=9 cPs, DDA=85% (by FTIR); Glacial acetic acid: Fisher Scientific, Catalog No. A38-212; Hydrochloric acid: 1.0 M aqueous solution Sigma Aldrich, Catalog No. H9892; Sodium hydroxide: 5.0 M NaOH aqueous solution Sigma Aldrich, Catalog No. S8263-150 ml; Ethanol: 200° Proof Decon labs Inc, Catalog No. 2701; De-ionized water: Ricca ACS Reagent Grade deionized water, Catalog No. 9152-5; 3,4-dihydroxyhydrocinnamic acid (Mw=182.17 g/mol): 98% Sigma Aldrich, Catalog No. 102601; 3,4-dihydroxyhydrocinnamic acid (HCA) (MW=180.16 g/mol: Sigma Aldrich, Catalog No. 822029; trans-3,4-Dihydroxycinnamic acid (trans-caffeic acid) (MW=180.16 g/mol): Sigma Aldrich, Catalog No. 51868; 3,4-Dihydroxyphenylacetic acid (DOPAC, Homoprotocatechuic acid) (MW=168.15 g/mol): 98% Sigma Aldrich, Catalog No. 850217; 1-ethyl-3-(-3-dimethylamino-propyl)-carbodiimide: (alternatively N-(3-dimethylaminopropyl)-N′-ethylcarbodiimide hydrochloride with common acronym EDC) Sigma Aldrich, Cat. #E7750; Iron (III) Chloride (Mw=162.2 g/mol): 97% Sigma Aldrich, CAS 7705-08-0; Sodium Chloride: Sigma Aldrich, Catalog No. 793566-500 g; Synthetic gastric solution: Pepsin-Sigma Aldrich P7000-25G, NaCl-Sigma Aldrich 793566-500G, H2O ACS Reagent grade, NaOH-Sigma Aldrich, Catalog No. S8263-150 ml; Tissue: fresh swine bladder mucosa, fresh swine stomach mucosa from Animal Biotech Industries Inc.; Citrated bovine whole blood: Lampire Biological Laboratory Bovine CPD, Catalog No. 7720010; Cyanoacrylate: Permabond 910 Tissue Adhesive, Catalog No. 72590; Dialysis Tubing: 3,500 Da MWCO Snakeskin Dialysis Tubing (Fisher Scientific), Cat. #PI88244; Polystyrene 150 mm×20 mm petri dishes: Celltreat Cat. #229655.

### Example 2

### CGHD Thin Dressing Preparation

CGHD freeze-dried, catechol-modified, porous-matrix chitosan dressing prototypes with thickness near 40-140 microns and density near 0.5-1.1 g/cm³ were prepared. Preparation was by freeze phase separation of 0.5% w/w aqueous catechol chitosan solutions at −40 to −50° C.; sublimation removal of ice by freeze-drying to provide dry matrix (≤4% residual water by mass); and matrix thermal compression at 40-80° C. to final thickness dressing sheet. Dressings were cut to size from compressed sheet. Electrospinning was briefly investigated as an alternative to provide nanofiber mat forms, but the spinning was too variable and with insufficient density to compete with the freeze-drying approach. The Pandalus borealis sourced chitosan (Cs) lots from Primex Iceland had number average molecular weight near 150 kDa and degree of deacetylation near 85%. The catechol chitosan (Cs-Cat) was prepared with a moderate degree of o-quinone crosslinking of the chitosan by pH control (in the range pH 5.3 to 5.7) of the Schiff base crosslinking reaction. The Cs-Cat was formed by N-acylation {approximately 25-30% in the presence of 1-ethyl-3-(3-dimethyl-aminopropyl)carbodiimide} substitution of the chitosan gluco-pyranose C-2 amine with 3,4-dihydroxyhydrocinnamic acid. Dialysis was performed at pH 6.0 and 6.3 with multiple dialysate changes to remove unreacted low molecular weight residue.

An important modification of the Cs-Cat synthesis was introduced to address lot to lot variability in the catechol modified chitosan and increase in N-acylation of the 3,4-dihydroxyhydrocinnamic acid percent addition to glucosamine C-2 amine to above 35%. The modification involved the addition of ferric chloride which assisted in N-acylation, promotion of Schiff base crosslinking, enhanced hemostasis, and significantly reduced inconsistency in dressing lot preparation. The Fe-modified Cs-Cat dressings were prepared using the same method as for the catechol modified dressing, but with the addition of FeCl₃ in either a 1:5 (Moderate) or 1:1 (High) molar ratio of Fe to catechol reactant moiety (CatH). A 1:15 (Low) addition of FeCl₃ addition was also investigated but found to provide for lesser and less reliable performance similar to no addition of FeCl₃.

Compositions of one, two or more layer thin dressings of iron-enhanced catechol chitosan (IECD), catechol modified chitosan and plain chitosan were prepared by adhering different composition uncompressed freeze-dried sponges together during compression under high load (≥10 Bar) and 80° C. Alternatively, multiple or single layer composition dressings were formed during the mold solution freezing step at −40 to −50° C. whereby a first (or single) layer was first formed by addition of 0.5% aqueous solution to the mold and freezing the 1 to 4 mm thick layer for 20 to 40 minutes; and then, if adding a further layer, pouring or spraying the different dilute solutions to form the next frozen layer on top of the previous layer. The preferred first layer solution was an iron-enhanced catechol chitosan (IECD) dressing since this provided the strongest and longest duration adherence to gastric mucosa under simulated, wet 37° C. gastric treatments. After freezing the single or composite structure in this way, the structure was sublimated to dryness (≤ 4% residual water by mass) by freeze drying. The low density (close to 0.005 g/cm³) freeze dried cake of single or composite layers was then compressed between heated (40-90° C.) platens to thickness≤140 microns and density ≥0.5 g/cm³ and final dressings were cut to their desired size. This dressing preparation approach enabled ready combination of targeted properties including foldability, resistance to swelling, resistance to gastric fluid digestion, tissue adhesion and mechanical strength.

The three dressing types of two-layer compositions (A, B and C) presented below (see Table 1) provide demonstration of the exceptional properties of the Fe Cs-Cat (IECD) composition when it is delivered with Fe Cs-Cat surface against the wound to quickly, safely and reliably control bleeding with reduced opportunity of rebleeding. The results of this testing are equally relevant to single layer Fe Cs-Cat and more than two layer forms of base (against the wound) Fe Cs-Cat dry dressings.

TABLE 1

| Name | Dressing Tissue Adhesive Layer | Dressing Backing Layer |
|---|---|---|
| Type A | High Fe Cs-Cat | Cs |
| Type B | Cs-Cat | High Fe Cs-Cat |
| Type C | Moderate Fe Cs-Cat | Cs |

Some variants in the Freeze-Drying method (dressing freezing/thawing and refreezing) were introduced whereby the dressing structure was changed from being substantially formed of thin lamella structure <5 microns thick oriented near 45° from the vertical to substantially structure at 90° to the vertical (horizontal structure). The dressings formed of horizontal structure demonstrated significantly lower flexural modulus compared to the predominantly 45° structure dressings. This lower flexural modulus in the horizontal structure dressings provided for improved ease of unfolding in delivery as well as excellent tissue conformance. Type B dressings in later batches of dressings were substantially formed from horizontal structure chitosan lamella.

Ethylene oxide (ETO) and gamma-irradiation (25-40 kGy) sterilization treatments were investigated for possible changes to the dressings. Gamma irradiation was found to cause minimal change (ultimate tensile strength of dressing ≥7 MPa) while ETO increased adhesion with loss of mechanical strength (ultimate tensile strength of dressings <7 MPa). Both ETO and gamma-irradiated sterilization groups were investigated in animal hemostasis studies with gamma irradiated sterilization (25-40 kGy) providing dressings with improved delivery and hemostatic properties compared to the same dressings ETO sterilized. All animal testing data provided in the specification examples was of gamma irradiated (25-40 kGy) samples.

### Example 3

### Prototype Wire Delivery Device Design and Development

Five iterations of an 8-wire, 4 arm nitinol prototype were designed and prepared. Tricol Biomedical explored the nitinol wire delivery basket using gastrointestinal nitinol stents formed into improvised basket shapes. A simple prototype of supported nitinol wire loop design was explored.

### a. 8-Wire, 4-Arm Nitinol Superelastic Prototypes

A superelastic, 4-arm, nitinol-wire prototype delivery system was designed and developed that, when folded, would constitute 8-wires with 4 loop ends housed in cross-section of the catheter delivery tube distal end. The prototypes were prepared from two sets of identical mold-constrained, salt-oven cured, nitinol wires (0.24 mm or 0.32 mm diameter). After heat-setting, these two wires formed two, separate, tapered “T” outline shapes with each wire end beginning and ending at the base of the T (with 2.5 mm distance between wire ends). The axis strut wires tapered to a 7 mm diameter central circle at the top of the “T” before extending apart to outline the base of the cross-strut. The structural elements of each heat shaped wire are i) the supporting axis-strut of the T and ii) the top cross-strut of the T at 90° to, and centered on the support axis-strut. The final device was formed by overlaying the two annealed T wire forms over each other and rotating one of the T's around the long, central T axis 90° and fixing the free wire ends to lock the final, symmetrical 3D conformation. Firm attachment of a dressing to the four corners of the cross-strut assists greatly with structural stabilization of the wire device. This attachment proved a challenge in the design of a system with a releasable dressing. The final device, looking from above down the axis-strut, has the top cross-struts of the T forming a —cross— with each arm at 90° to the other (i.e. the arms forming 4 prongs similar to the compass's East, West, North and South). In compression, within a narrow catheter such as target 2.4 and 3.2 mm internal diameter tubing (that can be deployed from around 2.8 mm and around 3.8 mm diameter gastroscope channels respectively), the four wires of the central axis strut of the wire device align with the tubing axis and are distal to the delivery end of the catheter. The end of the wire delivery device that is proximal to the catheter delivery end has the folded, 8-wire, central portion of the cross-struts extending distal to the catheter delivery end with the folded, 8-wire four-point extremities of the cross-strut arms extending proximal to the catheter delivery end. The main advantages of the folded 8 wire delivery system are i) low cross-sectional profile (near 0.44 mm²) of the 8 wire system inside catheter tubing such as a 2.4 and 3.2 mm ID (4.5 and 8.0 mm² respectively); and ii) relatively low cost in the nitinol wire delivery system (<$75 each in mass production cost).

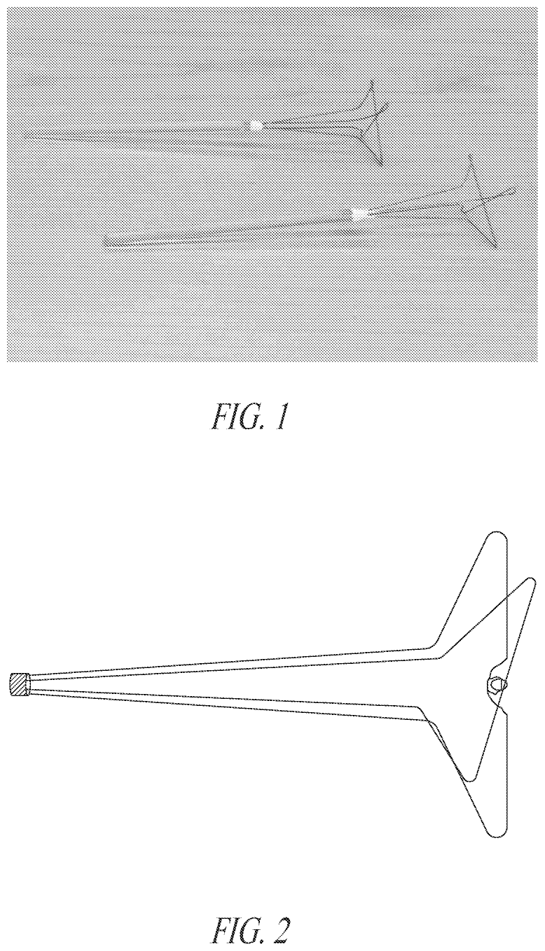

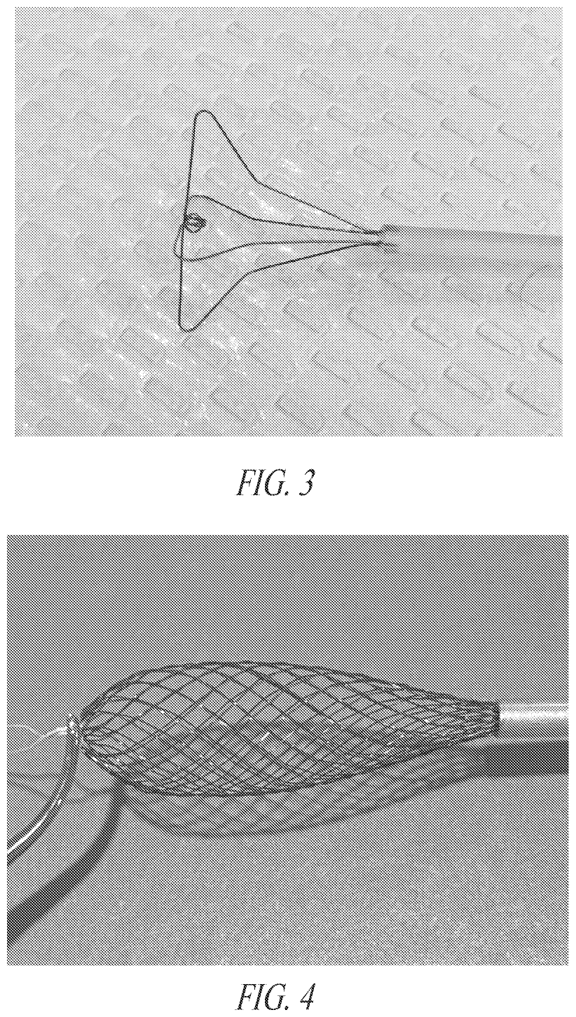

Of the 5 iterations of prototype development, the first included 3 devices formed of 0.24 mm diameter wire and 3 formed of 0.32 mm diameter wire with T central axis 40 mm and cross strut 20 mm. The four T cross-strut ends were each rotated 90° to provide for improved adhesion attachment to dressings (FIG. 1). This first prototype had a problem with sliding and catching of the crossed cross-struts during folding, compression and deployment from within a 3.5 mm internal diameter catheter tube. The second iteration of the wire device was very similar to the first (3 devices formed of 0.24 mm diameter wire and 3 formed of 0.32 mm diameter wire) however two centrally interlocking tight wire loops (each close to 1.5 mm in diameter) were included in center of the two cross-struts to articulate the cross-struts together centrally and to remove possibility of entanglement on deployment. Additionally, the four T cross-strut ends were not rotated 90° as it was found that this twist complicated wire shaping without obvious additional dressing attachment benefit. The third iteration of the prototype wire development (3 devices with 0.24 mm diameter wire) was performed to introduce larger diameter central wire interlocking loops (2.5 mm diameter) with one loop being recessed in the other to further remove wire catching issues during folding/delivery and to solve a problem of nitinol wire failure at the tighter loop in the second iteration. FIG. 2 provides a perspective drawing of the third prototype (FIG. 2). The third iteration was with central axis 40 mm and cross strut 20 mm. This third and later iterations of the prototype were all constructed with 0.24 mm diameter nitinol wire which provided for less “snap” spring force in delivery and thus less stress on the delivered dressing and its attachment to the wire device. Application of the opened 0.24 diameter, 4-prong device against the flat pan of a mass balance demonstrated elastic loading of the wire between 50-100 g of load mimicking the elastic loading during dressing delivery. Iterations four and five (3 prototypes each) of the delivery device development prepared a device with shorter axis-strut (reduced from 40 to 20 mm, see photograph in FIG. 3), one that could be loaded inside a 3.7 mm OD catheter (base wire locking attachment diameter <3.2 mm diameter) and one that better retained wire superelasticity.

b. Multi-Wire, Interwoven, Nitinol Superelastic Basket Prototypes

Samples of superelastic bilial and esophageal nitinol stents of cylindrical shape were used to prepare improvised wire basket prototypes able to deliver folded CGHD's from catheters. The stents were formed of 45° and 135€ (relative to stent cylindrical circumference) interwoven nitinol wire spirals with spacing at least 2 mm between adjacent wires. There were between 24 to 34 equally spaced wires around circumference. Wire diameter was either 0.22 or 0.18 mm. The cylindrical gastrointestinal stent diameter was between 10 mm to 22 mm with stent length from 55 to 85 mm. The improvised basket shape was formed (FIG. 4) by using braid suture to tie the delivery (proximal) end of the stent tightly closed. The support (distal) end of the stent was compressed, stretched and placed firmly inside appropriately sized support tubing. Aluminum tubing 4.0/3.2 mm OD/ID was found to provide reliably firm support allowing straightforward adjustment of the length of the uncompressed basket end of the stent without risk of wire damage. Smaller diameter support tubing such as stainless-steel cannula tubing was also considered, however to avoid excessive load on the stents, the 4 mm diameter aluminum tubing was elected for support and exploration of the basket design.

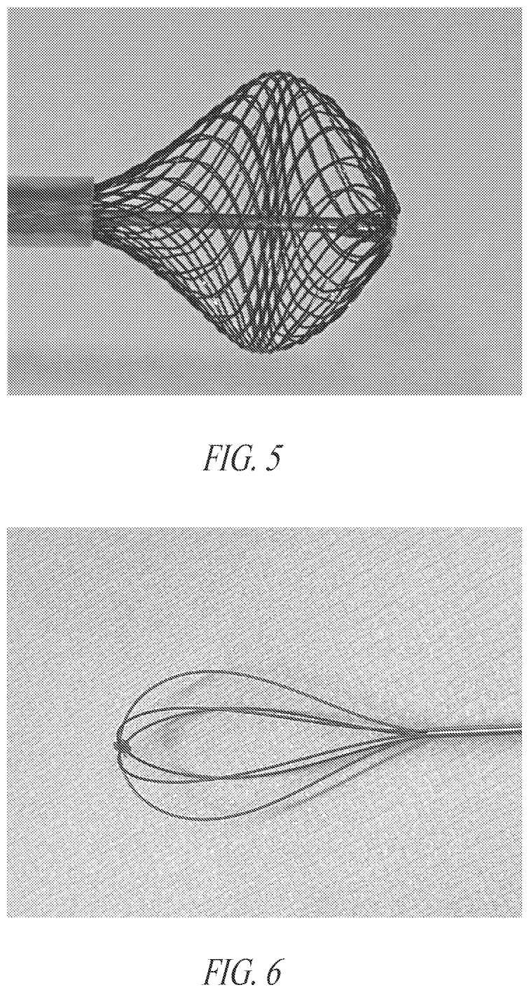

In exploration of dressing loading and delivery, the improvised stent basket supported on the 4.0/3.2 mm OD/ID aluminum tubing were paired inside 5.6/4.4 mm OD/ID polyethylene tubing. Smaller diameter tubing to 3.7/3.2 mm OD/ID was demonstrated as being feasible with this improvised design depending on the outer diameter of the basket support, the number of wires around the circumference of the stent and wire diameter. Testing of the improvised basket design was performed using two identical esophageal stents 20 mm diameter and 85 mm length with 0.22 mm diameter nitinol wire. The ends of the stents were closed tightly with a strong suture braid. 80 mm compressed length of the stent was placed inside the 4.0/3.2 mm OD/ID aluminum tubing which resulted in close to 80 mm length of compressed stent available to form the basket. A central rod formed of 1.3/0.9 mm OD/ID stainless steel cannula was attached through the middle of the compressed stent and its support aluminum tubing handle, fixing the proximal end of the rod with a tight loop of 0.34 mm diameter brass wire around two sides of the closed proximal end of the stent. The addition of this rod was used to assist with initiation of opening and rounding of the nitinol basket without need for any loading force against a target delivery surface such as the stomach wall mucosa. The rounded basket shape end (FIG. 5) was readily initiated by pulling on the cannula rod while pushing on the aluminum barrel. Once the round basket shaped end was established, subsequent loading of the basket end along its central axis demonstrated a spring tension of close to 250 g. The stiff central cannula rod also provided stability in axially loading the end of the basket without lateral basket movement. This sideways movement was found to cause difficulty in axially loading the 8-wire, 4-arm and looped wire prototypes. A final basket design would be heat shaped to automatically open the basket to a rounded shape without need for a central rod to assist in opening. Once opened using shaped elastic memory, a short-necked basket (length/diameter≤1.0) is less susceptible to lateral rocking during loading application providing even less need for the central rod device. Should some central lateral support still be desirable, then a stiff but flexible central rod material (e.g. 0.5 mm diameter nitinol or graphite wire) might be considered.

c. Looped Wire Prototypes

A simple third prototype nitinol wire loop design consisted of individual wire loops of diameter near 20 mm of either 2 looped wires offset at 90° to each other, or 3 looped wires offset at 60° (FIG. 6) with wire ends fixed in 1.3/0.9 mm OD/ID stainless steel cannula. This prototype lacked the beneficial properties of the 8-wire 4-arm device and basket delivery systems due to a spring load of >300 g and issues with lateral instability during loading. A very useful property of the simple loop is that it is readily prepared in number without great expense for use in accelerated shelf-life testing of loaded dressing opening.

### Example 4

### CGHD Dressing Wire Attachment and Release

### a. 8-Wire, 4-Arm Nitinol Superelastic Prototype

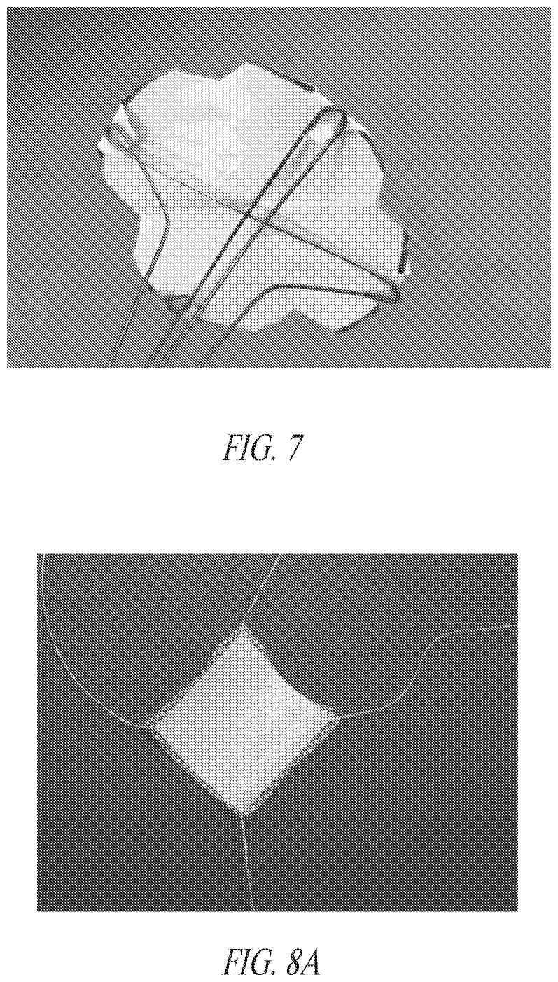

Attachment of catechol and iron-enhanced catechol modified CGHD dressings was initially investigated with cyanoacrylate adhesion to 3 to 4 mm length of outside wire strut through 4 articulating CGHD tabs 90° apart on its circumference (FIG. 7). Direct cyanoacrylate gluing adhesion of the chitosan freeze dried dressings to the nitinol wire proved problematic since the surface of the dressing was prone to delamination. Also stress at the dressing attachment tabs could also lead to tab tearing upon deployment spring action. A major issue with tab attachment was ensuring that all the tabs would soften and release their dressing corners within 30 seconds of exposure to blood so that the wire device could be removed leaving the dressing attached covering a bleeding injury site. Not all tabs were as equally accommodating, and another solution was required to provide not only reliable delivery device attachment but also reliable delivery device release.

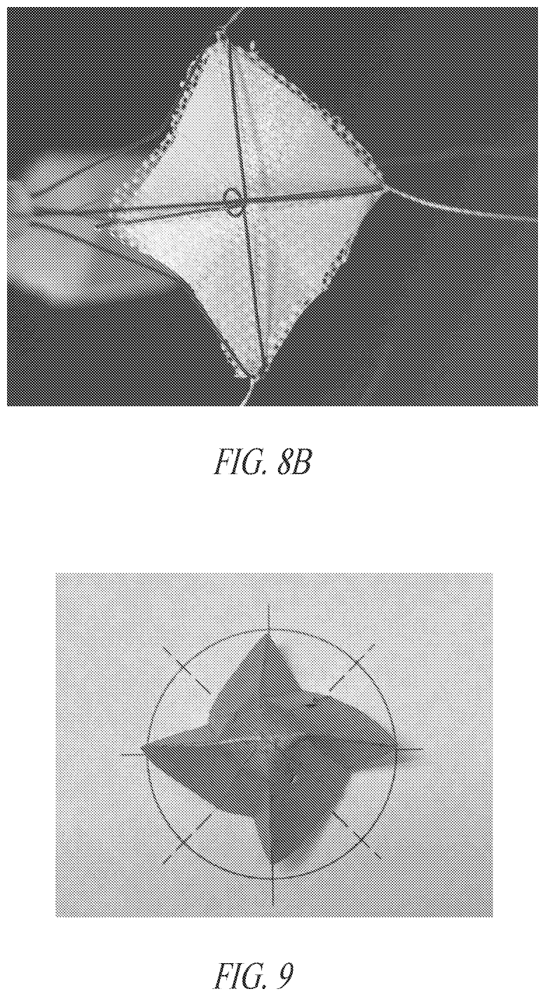

A preferred method of attaching the dressing was developed using blood dissolvable, polyvinyl alcohol (PVA) thread that dissolved fully within 30 seconds of wetting. We attempted to glue the PVA thread using cyanoacrylate glue, however this adversely affected the ability of the PVA to dissolve on wetting. The PVA thread method of attaching the CGHD dressings to the 20 mm long “T” cross-struts was first attempted with 20 mm diameter round dressings, and subsequently with 15 mm×15 mm square dressings. The square dressings proved easier to fold and load in a 3.5 mm ID catheter. The thread was used to tie four equally spaced corners along the edge of the CGHD dressings to the four corresponding equally spaced corners of the ends of the nitinol cross struts of the 8-wire+4 arm delivery prototype. The tie points were protected from slippage along the smooth nitinol wire by application of 2 closely spaced, small droplets of cyanoacrylate on the wire cross-strut ends. Once the pairs of droplets fully cured to form two firmly adhered small, raised dots, the PVA thread was tightly tied between them with loose thread removed by trimming. The PVA thread was adhered in place to the CGHD dressing using either heat welded Delnet polyethylene adhesive netting or a thin (<20 microns thick) 3M polyurethane elastomer (PUE) film with a thin layer of pressure sensitive 3M medical polyacrylate adhesive. FIGS. 8A and 8B showing Delnet backed CGHD dressing with PVA thread —untrimmed—attachment to the iteration 3 prototype). The Delnet attachment method proved to have variable adhesion to the PVA thread and the chitosan dressing therefore we concentrated our efforts on the PUE film. The PUE film reliably adhered and sandwiched the PVA thread from corner to corner of the back of the dressing along the 20 mm diagonal fold lines with close to 50 mm of free PVA thread available at each of the 4 dressing corners to provide for tying. A commercial CGHD with PUE backing would include a medical bioabsorbable polyurethane elastomer.

b. Basket and Looped Wire Prototypes

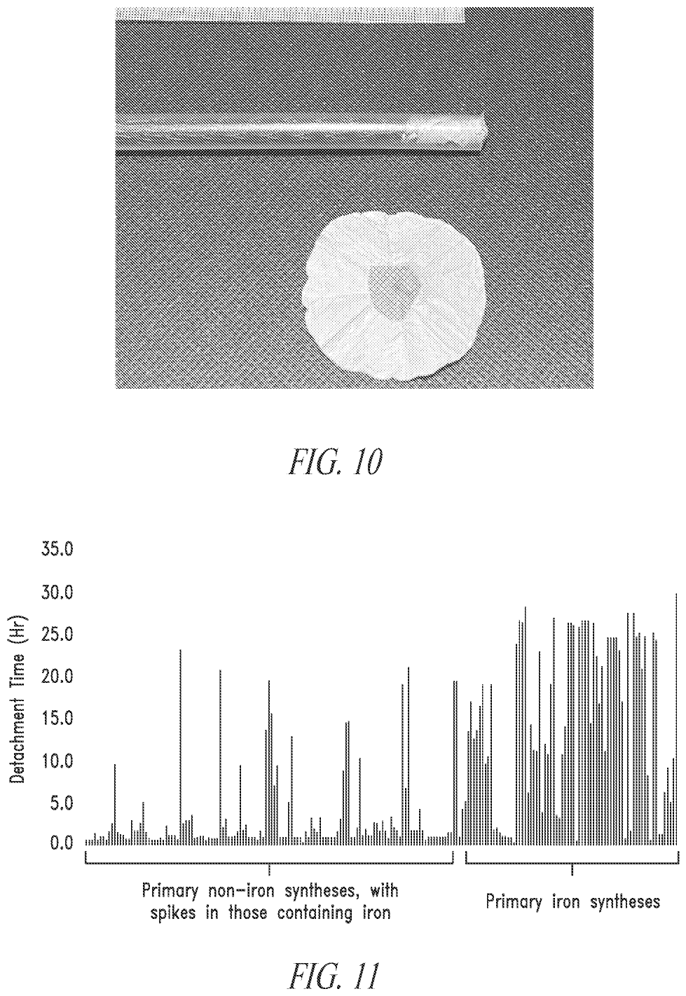

Dressing attachment and release from basket and looped wire prototype devices proved to be relatively straightforward since the CGHD dressings (FIG. 9) could be folded tightly around the compressed proximal ends of baskets and loops and then loaded under compression inside the proximal catheter end to be delivered by expressing the basket or loop with its dressing out of the end of the catheter tubing. The dressing may be attached to the proximal tip of the basket or wire with a small, tied loop of dissolvable PVA thread attaching the center of the dressing to the proximal wire tip. In application of the catheter delivery system with its CGHD dressing to quickly control bleeding to a targeted bleeding site, we found that use of the PVA thread was unnecessary in the prototype testing because targeting invariably rested the tip of the catheter against the target site before expressing and opening the dressing out of the catheter end. The spring loading of the expressed basket resulted in sandwiching the opened dressing accurately and firmly against the bleeding site with no chance of misplacement of the dressing. It was found that the sharp sewn end of the improvised nitinol baskets provided an initial risk of perforation of the middle of the dressing during the tight loading and delivery. Adherence of a small, thin, medical elastomer reinforcement circle of 3 mm diameter (FIG. 10) eliminated all perforation providing for highly reliable and effective dressing delivery by basket. Similar reliability was observed in loading and delivery from loop wire prototypes.

### Example 5

### In Vitro, Shelf-Life & Biocompatibility Testing (Non-Hemostasis CGHD Performance) of Type A & C Dressings

a. Methods
i. Beaker Test

A 38 mm×38 mm piece of fresh stomach mucosa is adhered to the base of a polystyrene beaker (250 ml, Fisher Catalog No. 08-732-124) using a thin layer of cyanoacrylate adhesive applied using a cotton swab. Close to two drops of synthetic gastrointestinal fluid are placed on the tissue mucosal surface during the cyanoacrylate curing to prevent tissue drying. The mucosa surface prior to gluing is dabbed dry using Texwipe tissue. The adhesive is allowed to dry over 2-5 minutes. After becoming fully adhered to the beaker, the top exposed tissue surface is wetted dropwise (generally 2 drops) with citrated whole bovine blood, and a 0.5″×0.5″ piece from a CGHD test article is adhered to the blood covered mucosa surface with application of 50 g/cm² pressure for 30 seconds (250 g of load applied orthogonally over test article on the mucosa surface through a 25 mm diameter PVC flat head probe/Probe is carefully removed from dressing surface avoiding any dressing detachment). After 30 minutes, synthetic gastric fluid at room temperature is added to the beaker with sufficient volume to just cover the patch. The system is observed for 15 minutes before the top of the beaker is sealed with Parafilm and the beaker is placed upright on an IKA KS260 orbital shaker in an incubator at 37° C. under mild shaking (130 rpm). The appearance of the dressing (including any dimensional changes) inside of the beaker is monitored at hourly intervals until test article separation from mucosa and its dissolution-/fragmentation. Dressing dimensional stability when wet was qualified as meeting acceptance for dimensional change on wetting (typically swelling) if there was no significant length×width changes to dressing attached to mucosa in beaker test that resulted in its detachment within 6 hours of wet mucosal application. Dressing dimensional changes and times to separation, dissolution, and/or fragmentation are recorded.

ii. Foldability

Test sheets (25 mm×25 mm) are folded 180° along length and width axes with fold crease lines folded tightly. The test sheets are unfolded and observed for visible tearing and cracking with recording of success being absence of significant cracks or tears.

iii. Mechanical Testing to Failure

A uniaxial mechanical tester (Instron 5844) with 2 kN load cell was used to investigate dry dressing ultimate tensile strength (UTS) and percentage (%) elongation to break. ASTM D638 was used guide design of the testing method. Pneumatic grips with grip pressure of 20 psi were used to grip adhesive taped top and bottom (0.5″×0.5″) of 0.5″×2″ samples. Thickness of each sample was determined prior to each test using calipers. The crosshead speed in testing was 5 mm/min. Ultimate tensile strength (MPa) was determined from maximum load and sample thickness. Gauge length was set as 1.00″. Percentage elongation to break was determined as a % change in gauge length.

iv. Mechanical Testing for Adhesion

A uniaxial mechanical tester (Instron 5844) with 10 N load cell was used to investigate wet adhesion to mucosa. Adhesion testing was performed using ASTM F2258-03 “Standard Test Method for Strength: Properties of Tissue Adhesives in Tension”. Testing was performed with a testing configuration with lower and upper PVC probes uni-axially aligned in the z vertical direction so that the edges of their x-y horizontal, 15.2 mm diameter faces would accurately (+0.2 mm) coincide with each other with uniaxial lowering of the top probe which was supported on the upper, movable Instron crosshead in chuck fixture. The lower PVC probe was supported in a stationary, bottom, chuck fixture. The bottom PVC horizontal surface was used to support a 10 mm×10 mm mucosal tissue sample adhered at least 5 minutes before testing by cyanoacrylate glue to the PVC surface. The top PVC horizontal surface was used to support a 10 mm×10 mm CGHD test piece that was adhered by a 3M double side tape at least 5 minutes before testing The square tissue piece was wetted with 0.25 ml of the de-citrated bovine whole blood CPD prior to lowering the probe onto the test surface. The probe was lowered at 10 mm/min until a maximum load of 0.98 N was reached. At contact, the test and tissue pieces contacted accurately (±0.2 mm) and were mutually co-planar. The uniaxial probe load at 0.98 N was maintained for 30 seconds after which the probe was removed at 10 mm/min and maximum failure stress was recorded.

v. Shelf-Life

Twelve month stability (assuming ambient=23° C. & Q10=2) of CGHDs was investigated in accelerated testing at 45° C. according to ASTM F1980 with acceptance based on meeting visual inspection and beaker testing.

vi. Biocompatibility

Cytotoxicity, direct contact irritation and 7 day implantation (upper gastrointestinal tract) biocompatibility testing of type A and C CGHD dressings was performed according to ISO 10993-1.

b. Results

The results are summarized in Table 2. Iron-enhanced CGHD dressings were able to be tightly folded, and unfolded from accordion-like forms without cracking or tearing. Iron-enhanced CGHD dressings demonstrated target wet adherence to gastric mucosa with swelling in gastrointestinal fluid at 37° C. less than 25%. The iron-enhanced CGHD dressings in synthetic gastric fluid at 37° C. demonstrated good adhesion to stomach mucosa, and strong resistance to dissolution at less than 12 hours, with complete dissolution before 168 hours. FIG. 11 shows compiled testing results of mucosa detachment time (FIG. 11).

TABLE 2
CGHD Dressing Performance

| CGHD Dressing Performance Specification | | Observation |
|---|---|---|
| Dissolution stability ≥12 hrs in synthetic gastric fluid at | | Meets specification |
| 37° C. | | |
| No cracking or tearing during folding and furl/unfurl | | Meets specification |
| Ultimate tensile strength of dry dressings ≥7 MPa | | Meets Specification |
| Swelling of dressing when wet | | Meets specification |
| Wet adherence to gastric mucosa ≥5 kPa | | Meets specification |
| Complete dissolution in gastric fluid at 37° C. in less than | | Meets specification |
| 168 hours | | |
| Delivery by WD (wire delivery) - able to furl/unfurl | | Meets specification |
| Delivery by WD - able to apply CGHD with release to ex | | Meets specification |
| vivo surface (no bleeding and bleeding) | | |
| Reproducibly manufactured in multiple lots | | Meets specification |
| 1 Year Shelf-life stability | | |
| a. | Visual Inspection (No change) | Meets Specification |
| b. | Beaker test | Meets Specification |
| ISO 10993-1 Biocompatibility: Cytotoxicity, irritation, | | Meets Specification |
| acute systemic toxicity, implantation | | |
| a. | Cytotoxicity ISO Agarose Overlay | Passed: Nontoxic |
| b. | Primary Skin Irritation | Passed: Negligible irritation |
| c. | Swine Upper Gastrointestinal Application | Passed: No adverse responses at |
| | to Injured (6 mm biopsy punch) Mucosa | 72, 144, & 168 hours. All |
| | (Implantation) | wounds demonstrating normal |

healing (non-inferior to
untreated control).

### Example 6

Wire Delivery of CGHD to Control Upper Gastrointestinal Bleeding (UGIB) in an Acute Swine Injury Model of Forrest 1a Hemorrhage (Study 1) a. Method

The objectives of the acute study were:
i) Investigation of ease and accuracy of delivery for the two different delivery methods and their dressings.
ii) Investigation of the time to hemostasis for the dressings and their delivery methods.

All animal work was approved by Oregon Health and Science University under Animal Welfare Assurance Number A3304-01. A midline laparotomy was performed on anesthetized, heparinized (ACT≥250 s) domestic swine, 40 to 50 kg, to expose the stomach. To create a brisk UGIH model (Forrest Ia), a gastrostomy was performed along the external surface of the greater curvature of the stomach and a 5-cm left and/or right gastroepiploic bundle was dissected and placed into the stomach cavity, and the gastric mucosa and seromuscular tissues were re-approximated over the vascular bundle. The lumen of the gastric cavity was exteriorized through a 12 cm incision along the anterior gastric wall. A pulsatile bleeding injury was created by partial transection of the vascular bundle.

In the hemostatic testing, the same injury was able to be reused if the surgeon believed the immediate prior location continued to demonstrate a sufficient rate of bleeding (>5 g/min), otherwise a new injury was created. Fresh injury creation was most often the case as the chitosan dressing application generally promoted strong clotting around the injury site such that when a dressing was removed after 5-10 minutes there would often be no further bleeding. Before application of a new dressing to a re-used site or a new site close to the immediate prior site, the surgeon or surgical assistant lavaged the field with 0.9% saline solution followed by wiping the area gently (avoiding disturbing the mucosa) to remove any residual chitosan.

CGHD dressing type A, B, C, See Table 1, Example 1) in combination with non-Basket and Basket wire delivery devices were tested by the surgeon and surgical assistant(s) in the open-stomach, Forrest 1a swine injury model (2 swine). The dressing thicknesses were between 100 microns and 120 microns. Basket delivered dressings were all 25 mm diameter and circular in shape. The non-Basket wire delivery device dressings were all 15 mm×15 mm square dressings. Delivery of the folded CGHD dressing to the bleeding injury was through a 4.4 mm diameter channel.

The activated clotting time (ACT) of the swine was maintained above 250 seconds using 0.5 unit/ml heparin solution intravenous drip at 250 ml/h rate. If ACT falls below 250 see, an additional 1000 unit of heparin would be given IV immediately to boost ACT above 250. All bleeding injuries were used in the study with bleeding rate found to vary from oozing and spurting 1 to 30 g/min with the majority of bleeding >5 g/min. After a 30 see tamponade dressing application (100-250 g/cm²), successful hemostasis was assessed as substantial absence of visible bleeding within 3 mins of dressing application. Inability to control bleeding after 3 minutes of dressing application was rated as failure.

At the conclusion of successful testing, all CGHD dressing types were ranked for tissue adherence by the surgeon before dressing removal. The dressing tissue adherence ranking is provided below:
0=no adherence (dressing may wet tissue surface but there is no detectable binding),
1=low adherence (some adherence but with minimal effort to lift edge)
2=low to moderate adherence (need to use moderate force to lift an edge)
3=moderate to strong adherence (at least one to two edges resist removal, with other edges adhering moderately)
4=strong adherence (all edges grip securely with all firmly resisting lifting).

At the conclusion of the tests on, the last dressing (Type A) was left adhered in place on the injury site and one Type B dressing and one Type C were adhered with and without a small amount of blood on the stomach mucosa adjacent to the Type A dressing adhered over the injury. The swine was then sacrificed shortly after and the whole stomach with attached dressings was surgically removed and bundled into a zip-lock bag and left for investigation of the dressings the following day at room temperature in the laboratory.

b. Results

There were 31 test applications with dressing types A, B and C using Basket and non-Basket delivery methods. Dressing delivery accuracy to the focal bleeding location was judged to be close to ±3 mm for both types of dressing delivery making the difference in dressing size between basket and non-basket inconsequential. The average (±standard deviation) injury bleeding rates immediately prior to type A, B and C dressing applications were 8.4±8.3 9.78±11.9 and 7.61±6.9 g/ml respectively. The animal vital signs remained stable over the period of testing which was close to 7 hours on both days. Over all the testing, successful hemostasis was ≥75%, with >45% of the applications demonstrating hemostasis within 30 seconds. Dressing type B, the most flexible and best wound conforming dressing, demonstrated 100% (9/9) successful hemostasis in under 3 minutes with 8 basket and 1 non-basket applications.

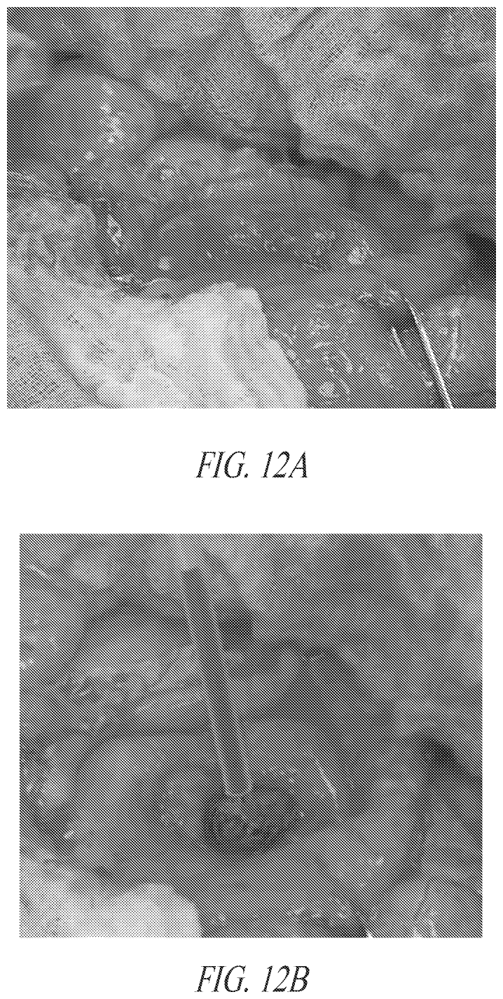

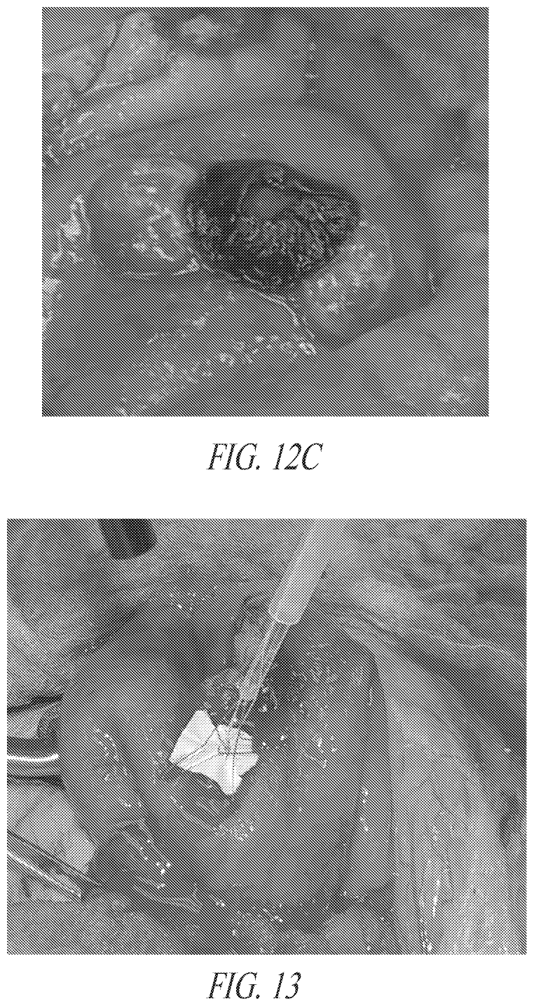

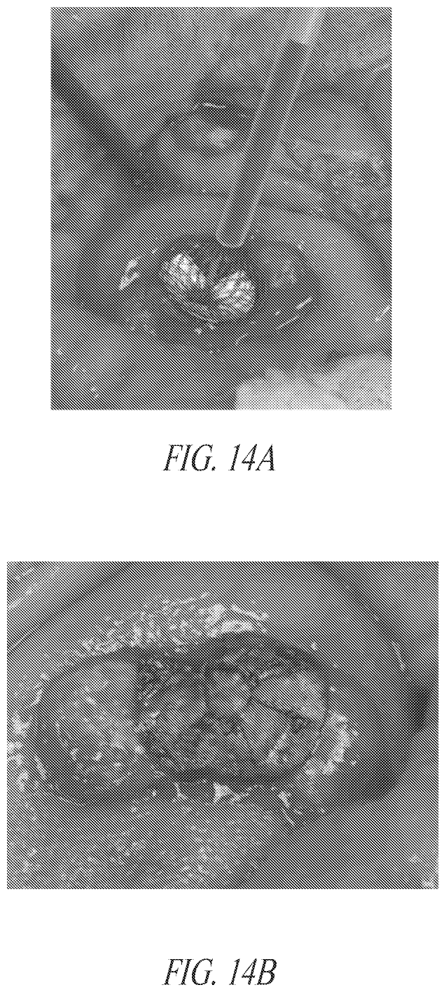

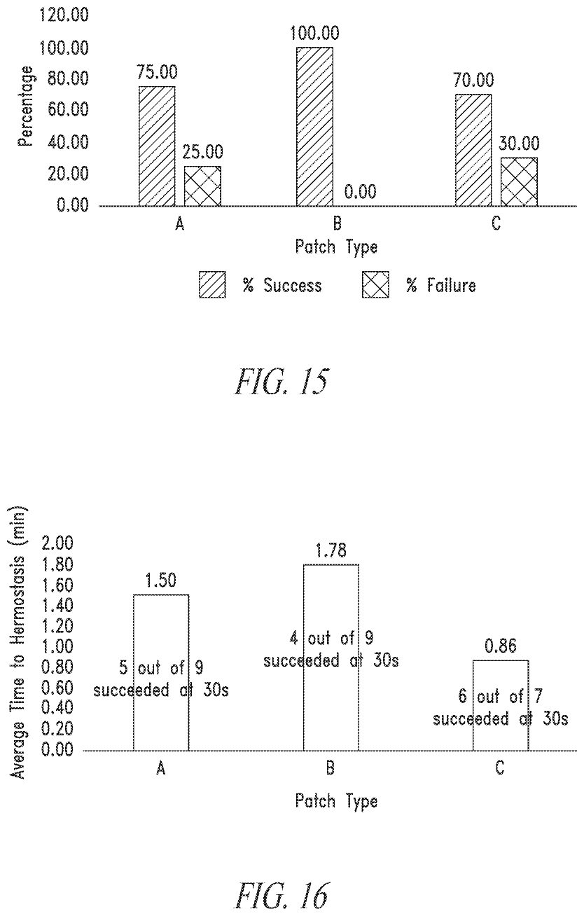

FIGS. 12A, 12B and 12C show the original untreated injury; basket delivery of Type B dressing to the injury; and successful dressing promoted hemostasis following Type B attachment over the injury (FIGS. 12A-12C). FIG. 13 demonstrates injury application by the non-basket wire delivery device of a Type C dressing (FIG. 13). FIG. 14A and FIG. 14B show basket delivery and successful dressing tissue attachment after delivery of a Type A dressing (FIGS. 14A-14B). FIG. 15 provides histogram results of the hemostasis success/failure for dressing types A, B and C (FIG. 15). FIG. 16 provides histogram results of time to hemostasis for dressing types A, B and C (FIG. 16).

There were 21/24 Basket and 4/7 non-Basket delivery device applications with successful control of bleeding within 3 minutes of delivery with average (±std) time to hemostasis of 1.2±0.4 and 1.8±1.3 minutes respectively (see Table 2). The basket delivery device proved more efficient and consistent compared to non-Basket device in dressing loading and delivery. Also, basket delivery device applications demonstrated greater and more uniform adherence to stomach mucosa and externalized arterial vessels located inside the stomach. This enhancement of dressing tissue adherence associated with use of basket delivery was caused by the more uniform and consistent axial loading able to be delivered by basket application. The enhanced basket delivery combined with ability to reload the basket system quickly (within 2 minutes) was realized early in the testing. An additional benefit of the basket device is that the proximal end of the delivery catheter can be accurately placed at the bleeding site under gastroscope visualization before delivery of the dressing immediately at the site, whereas the non-basket device must be opened (obscuring the site) away from the injury with subsequent loss of injury site gastroscope targeting and visibility.

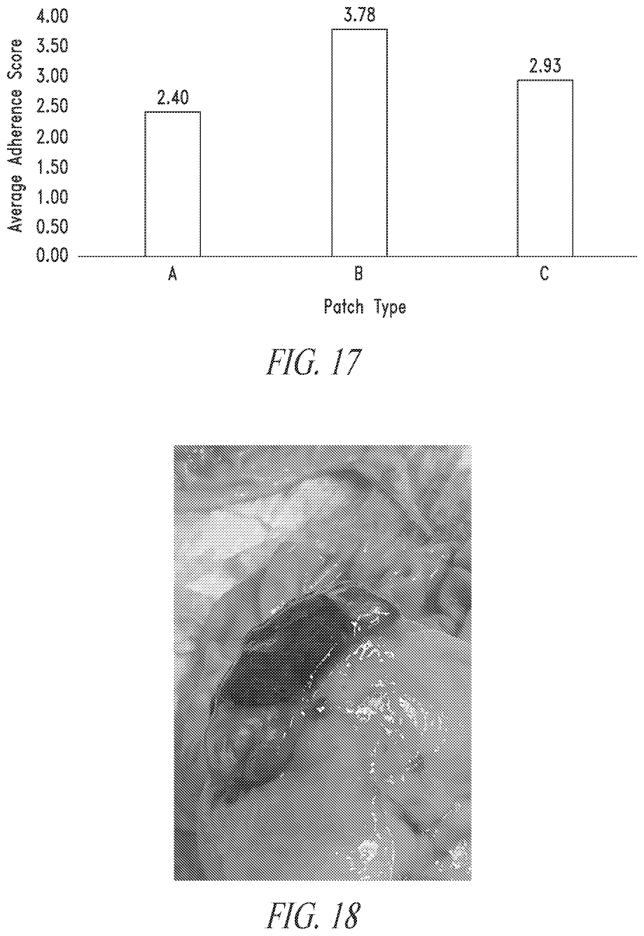

Adherence ranking results for the Type A, B, and C dressings are shown in FIG. 17(FIG. 17). It can be seen that Type B dressings that demonstrated 100% efficacy in control of bleeding also showed the highest level of tissue adhesion with an adherence rank of 3.8 (close to the ideal 4.0 rank). Types A and C demonstrated adherence ranks of 2.4 and 2.9 respectively.

The three type A, B and C of dressings which were left in place on the surgically removed stomach after animal sacrifice were demonstrated to be little changed the following day after 20 hours. The Type A dressing adhered over the injury the day before was shown to remain firmly adhered (adherence rank 4) to the vascular injury site (FIG. 18). The other two Type B and C dressings were also in place on the stomach mucosa demonstrating little change. The Type B dressing which was adhered with blood was more firmly adhered (adherence rank 3) than the Type C dressing applied directly to the mucosa without blood (adherence rank 2).

EtO treated dressings showed 100% hemostasis for total 6 applications with stent device, though only 17% applications reaching hemostasis within 30 seconds and time to hemostasis 3.3 (±0.5) minute was comparatively longer (data not shown). In addition, EtO treatment caused decrease in foldability.

TABLE 3
Comparison of basket and non-basket WD devices for
success and failure with dressing types A, B and C

| | | No of | | |
|---|---|---|---|---|
| Dressing Type | Device used | applications | Success | Fail |
| A | Basket | 10 | 7 | 3 |
| | Non-Basket | 2 | 2 | 0 |
| B | Basket | 8 | 8 | 0 |
| | Non-Basket | 1 | 1 | 0 |
| C | Basket | 6 | 6 | 0 |
| | Non-Basket | 4 | 1 | 3 |

The dressing prototypes of the invention have been demonstrated to be effectively delivered by prototype wire delivery systems through 4.4 mm internal diameter catheter tubing for ≥75% (all three types) and >90% (Type B) acute control of anticoagulated swine model Forrest 1a (major milestone 1) bleeding in less than 3 minutes.

### Example 7

Wire delivery of CGHD to control Upper Gastrointestinal Bleeding (UGIB) in an Acute Swine Injury Model of Forrest 1a Hemorrhage (Study 2)

The objective of the Example 7 study was to compare the hemostatic efficacy between Type A, B & C CGHD dressings using the basket delivery method alone, and previously represented in Example 6. The Type A, B & C dressings Lots used in Example 7 were prepared 8 months after those used in Example 6. The study was performed in the same swine model of anticoagulated bleeding in the upper gastrointestinal tract (Forrest 1a & 2a type bleeding) presented in Example 6.

Out of 45 applications total with 15 applications per type of hemostatic device, 13 out of 15 Type A applications were successful, 11 out of 15 Type B applications were successful, and 12 out of 15 Type C applications were successful. Out of 15 applications per device type Type A had 6 applications that achieved hemostasis within 30 seconds, Type B had 6, and Type C had 3 applications achieving hemostasis within 30 seconds. Type A had an average time to hemostasis of 108±87 seconds, Type B had an average time to hemostasis of 106±88 seconds, and Type C had an average time to hemostasis of 104±71 seconds. There were no significant differences in bleed rates, times to hemostasis, or hemostatic success between the three CGHD dressing types.

Success criteria for the test articles was ability to control bleeding with a combined hold time of at most (30+180 seconds) 210 seconds with either no observation of bleeding or a significant reduction in bleeding, as decided by the surgeon. The dressing unable to control bleeding by a total of 210 seconds is designated as a failure. Table 4 summarizes the results of this study.

TABLE 4
Summary of the results of Study 2

| | Type A | Type B | Type C |
|---|---|---|---|
| Number of Applications | 15 | 15 | 15 |
| Number of Successes | 13 | 11 | 12 |
| Number of Failures | 2 | 4 | 3 |
| Average Bleed | 5.25 ± 2.62 | 6.09 ± 2.88 | 6.42 ± 3.03 |
| Rate (g/min) | | | |
| Average Time to | 108 ± 87 | 106 ± 88 | 104 ± 71 |
| Hemostasis (s) | | | |

### Example 8

Manual Application of CGHD to control 6 mm Punch Biopsy Upper Gastrointestinal Bleeding (UGIB) in a Chronic Swine Injury Model

The objectives of the chronic study were: to investigate:−i) manually applied treatment CGHD dressings to Forrest 1b (6 mm punch biopsy) heparinized UGIB bleeding sites with open surgery; ii) surgical closure of the open stomach and laparotomy with resuscitation and monitoring of the 4 swine with sacrifice at 3 days (1 swine) and 6-7 days (3 swine); iii) retrieval and examination of the injury sites for demonstration of presence of treatment dressings and of non-inferior healing of the treated versus control (untreated) wounds.

Pre-Surgery Preparation: Veterinary staff inspected all of the animals to ensure baseline health. Animals were removed from all bedding 72 hours prior to the procedure and not permitted food 24 hours prior to surgery. Animals were allowed to drink water ad libitum. Twenty minutes prior to operation, the animals were given 500 mg of intravenous Cefotetan and a 250 ml fluid bolus of Ringer's Lactate. After premedication with glycopyrolate and a combination of tiletamine HCl and zolazepam HCl (Telazol®, Fort Dodge Laboratories, Fort Dodge, IA), anesthesia was induced by mask using 5% isoflurane. The swine were intubated, placed on a ventilator, and maintained with 2-3% isoflurane with endotracheal intubation. Vital indices, O2 saturation, CO2 tension, EKG and blood pressure were monitored during surgery. Preoperative lab studies included complete blood count (CBC) and chemical penal. Surgery area was shaved, prepped and draped in a sterile fashion.

Creating UGIB model: The swine were prepped using Chlorhexidine and draped in a sterile fashion. A midline laparotomy was performed to expose the stomach. An approximate 5-cm long incision was made on anterior gastric wall into gastric lumen. Posterior gastric lumen was exposed and the gastric bleeding injury was created through a 6-mm diameter tissue biopsy punch to remove the gastric mucosa layer. Bleeding rate from the gastric mucosa was determined by measuring mass of blood absorbed in 15-30 second using pre-weighed gauze.

In order to induce coagulopathy, 5000 units of heparin, prior to making the stomach incision, was given intravenously (IV). Activated clotting time (ACT) was determined after 10 minutes and then every 20 minutes during the procedure with additional heparin (50% of the original dose, 2500 units) given by IV as needed to maintain the ACT>250 seconds.

Chitosan Gastric Hemostatic Dressing (CGHD)) Treatment Application: The CGHD was laid onto the wound site of gastric lumen. As needed, a piece of non-stick spunbond polyolefin sheet was placed to cover lumen side of surface of CGHD to prevent adherence to the applicator. The applicator was a nitinol basket that uniformly distributed 50 to 100 g/cm² of applicator load over the CGHD. The applicator was placed with sufficient manual pressure to compress the nitinol basket on the top of CGHD or non-stick spunbond sheet if used, for 30 seconds. The applicator was carefully removed after 30 seconds without disturbing CGHD placement. After application of the CGHD to the bleeding site, the site was monitored for 3 minutes. If bleeding recurred within 3 minutes, a second CGHD was allowed to be applied. A total of 3 applications were allowed per treatment with possibility of CGHD overlapped on top of an earlier application or the earlier removed with application of a new dressing by itself. If hemostasis was not achieved within 3 applications, the treatment application was ruled as unsuccessful. Three sample treatments and one control (use of gauze alone held on the wound for 30 seconds & then removed) were used per animal. Generally, the gauze control sites demonstrated continuous low bleeding (hemostasis not achieved) after gauze removal. At closure, the control sites were judged to demonstrate sufficiently low bleeding to allow surgical closure. At the conclusion of sample treatment and control, if there was no significant bleeding after a final 10-mintue observation, the gastric incision was closed in a standard manner along with the abdominal wall. The animal was recovered from anesthesia and placed in the pen for up to 6 days observation. During follow-up, the animals were monitored for signs of post-operative complications, such as GI bleeding related shock, diarrhea, melena (recurrent bleeding) and discomfort. The swine were euthanized at end of study. Necropsy was performed for gross examination and tissue specimen were collected for histological examination.

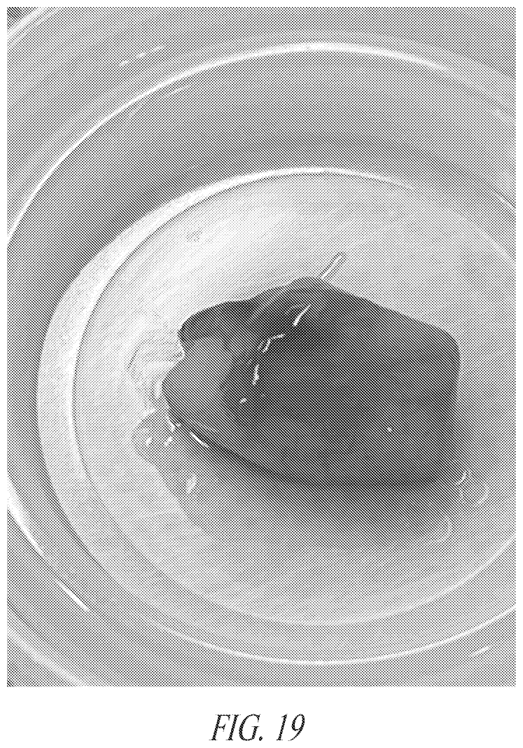

Result Summary: All 12 samples (6× Type A & 6× Type C) were implanted successfully controlling bleeding within 3 minutes of dressing application. The time of CGHD implantation for 1st, 2nd, 3rd and 4th animals was 3, 6, 7 & 7 days respectively. All animals demonstrated normal diet and behavior post implant. On necrosis, all implant sites demonstrated normal tissue healing. One Type A dressing was found still attached on its wound on the day 3 implant (FIG. 19) while there was evidence of Type C dressing residue in one Type C application (as indicated by backing material well attached to wound but no demonstrable mass of dressing). In the case of necrosis of the 6 and 7 day implanted animals, there were no dressings nor dressing residues found at the implant sites or close to the implant sites.

TABLE 5
Summary of results of chronic implantation

| | | | | | Dressing |
|---|---|---|---|---|---|
| | | | | | on Wound |
| | | | | | at explant |
| | | | | Normal | (Y/N/ R = |
| | | | hemostatic | healing | evidence of |
| Animal | Dressing | Implantation | success | on explant | dressing |
| # | Type | (days) | (Y/N) | (Y/N) | residue) |
| 1 | C | 3 | Y | Y | N |
| 1 | C | 3 | Y | Y | R |
| 1 | A | 3 | Y | Y | Y |
| 2 | A | 6 | Y | Y | N |
| 2 | A | 6 | Y | Y | N |
| 2 | C | 6 | Y | Y | N |
| 3 | C | 7 | Y | Y | N |
| 3 | A | 7 | Y | Y | N |
| 3 | A | 7 | Y | Y | N |
| 4 | C | 7 | Y | Y | N |
| 4 | C | 7 | Y | Y | N |
| 4 | A | 7 | Y | Y | N |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/208,209, filed on Jun. 8, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A releasable iron-enhanced chitosan dressing comprising an iron-enhanced catechol modified chitosan, wherein the dressing is hemostatic dressing.

2. The iron-enhanced chitosan dressing according to claim 1, wherein the iron is Fe^{III} or ferric chloride (FeCl₃).

3-4. (canceled)

5. The iron-enhanced chitosan dressing according to claim 1, wherein the dressing has a thickness that is about 40-140 microns.

6. (canceled)

7. The iron-enhanced chitosan dressing according to claim 1, wherein the dressing has a density that is in the range of about 0.5 g/cm³ to about 1.1 g/cm³.

8. (canceled)

9. The iron-enhanced chitosan dressing according to claim 1, comprising an adhesive side, a non-adhesive side, and a porous surface.

10. The iron-enhanced chitosan dressing according to claim 9, wherein the adhesive side is provided on a first layer and the non-adhesive side is provided on a second layer.

11. The iron-enhanced chitosan dressing according to claim 9, wherein the adhesive side adheres to a tissue surface when the dressing is wet, and wherein the non-adhesive side does not adhere to a delivery device when the dressing is wet.

12. (canceled)

13. The iron-enhanced chitosan dressing according to claim 11, wherein the dressing is configured to adhere to a gastrointestinal mucosa in 1 minute or less.

14. (canceled)

15. The iron-enhanced chitosan dressing according to claim 9, wherein the porous surface provides one or more of: (i) an absorbent surface; and (ii) channels to redirect moisture away from a target tissue surface site.

16. The iron-enhanced chitosan dressing according to claim 15, wherein the dressing adheres to tissue when the dressing is wet, and wherein the dressing adherence strength is greater than or equal to about 1 kPa.

17. (canceled)

18. The iron-enhanced chitosan dressing according to claim 1, wherein the dressing resists dissolution in water, saline solution, blood, or GI fluid at about 37° C. for at least about 6 hours.

19. The iron-enhanced chitosan dressing according to claim 1, wherein the dressing can be folded, punctured, sewn, furled, or any combination thereof, without cracking or tearing.

20. The iron-enhanced chitosan dressing according to claim 19, wherein the dressing, when in an open configuration, the open configuration being, unfurled, or unfolded, has an outward facing surface area that is one of: about six times greater, about five times greater, or about four times greater than the outward facing surface area of the dressing when in a closed configuration, the closed configuration being, furled, or folded.

21-22. (canceled)

23. The iron-enhanced chitosan dressing according to claim 1, wherein the dressing is able to be delivered intact by a delivery device, the delivery device being selected from a balloon device, a wire device, and an endoscopic device, the delivery device including a working channel having a diameter of 3.8 mm or less, through which the dressing is delivered.

24. (canceled)

25. The iron-enhanced chitosan dressing according to claim 1, wherein the dressing, when wet, is able to adhere intact to gastric mucosa in less than 30 seconds with application of light pressure of about 50-100 g/cm², and, optionally, wherein the dressing is able to remove hydrophilic and hydrophobic biological fluids that can interfere with adhesion.

26-27. (canceled)

28. The iron-enhanced chitosan dressing according to claim 25, wherein the dressing is able to stay in place intact and stop bleeding, the bleeding being from between about 1 g/min to about 30 g/min; or 2) greater than about 5 g/min.

29. (canceled)

30. The iron-enhanced chitosan dressing according to claim 25, wherein the dressing is able to resist dissolution for at least six hours after adhering to an injury site in presence of corrosive enzymes and acid environment of about pH 3, and wherein dissolution of the dressing does not exceed seven days.

31. The iron-enhanced chitosan dressing according to claim 15, wherein the dressing is able to seal and protect the target tissue surface site for a period of time of up to about 12 hours, or for a period of time from about 12 hours to about 96 hours.

32-34. (canceled)

35. The iron-enhanced chitosan dressing according to claim 1, wherein the dressing is not readily soluble in water, saline solution, blood, or GI fluid at about 37° C. for at least 12 hours following application.

36-37. (canceled)

38. The iron-enhanced chitosan dressing according to claim 1, wherein the dressing does not increase or decrease by more than about 25% in length and width, or more than about 50% in thickness, when in the presence of water, saline solution, blood, or GI fluid at about 37° C.

39. (canceled)

40. The iron-enhanced chitosan dressing according to claim 1, wherein the dressing is capable of being terminally sterilized and stored under controlled conditions without affecting dressing characteristics, wherein tensile strength is a dressing characteristic.

41-42. (canceled)

43. The iron-enhanced chitosan dressing according to claim 1, wherein the dressing is able to be used in the treatment of a disease, condition, disorder, trauma, or injury.

44-49. (canceled)

50. A method of producing an iron-enhanced chitosan dressing, the method comprising steps of (a)-(e):
(a) performing synthesis with chitosan and catechol in an aqueous reaction solution;
(b) maintaining a pH of the reaction solution at or below pH 5.5;
(c) after step (b), increasing the pH of the reaction solution, and controlling oxygen exposure to the reaction solution, to provide catechol oxidation and cross-linking;
(d) adding an aqueous solution of iron salt; and
(e) drying the reaction solution,
wherein the method does not comprise an intermediate drying step between step (b) and step (c), and wherein the iron salt comprises ferric chloride; or
the method comprising steps of (i)-(v):
(i) freeze-drying a first aqueous solution comprising catechol modified chitosan;
(ii) freeze-drying a second aqueous solution comprising catechol modified chitosan and an iron salt;
(iii) obtaining a low-density chitosan dressing with inter-connected porous structure from each of steps (a) and (b); and
(iv) compressing the low-density chitosan dressing from each of steps (a) and (b); and
(v) preparing a two-layer iron-enhanced catechol chitosan composite dressing from the compressed low-density chitosan dressing from each of steps (a) and (b).

51. The method according to claim 50, wherein, in step (c), the pH of the reaction solution is increased to a range of about 5.8 to about 6.2, and wherein, step (d) assists in promotion of Schiff base crosslinking, enhances hemostasis, and reduces inconsistency in dressing lot preparation.

52. (canceled)

53. The method of claim 52, wherein the iron salt is ferric chloride, and wherein the molar ratio of Fe to catechol reactant moiety (CatH) in the second aqueous solution is either about 1:5 (Moderate Fe) or about 1:1 (High Fe).

54. A gastrointestinal delivery system comprising:
(a) a wire delivery device; and
(b) a releasable wound dressing;
wherein the gastrointestinal delivery system is capable of fitting through a channel of about 4.4 mm internal diameter (ID) or less, and wherein the wire delivery device is capable of: (i) attaching to the dressing during loading; and (ii) releasing the dressing during delivery.

55-100. (canceled)
